(19) United States
(12) Patent Application Publication
(10) Pub. No.: US 2016/0166185 A1
(43) Pub. Date: Jun. 16, 2016

(54) BLOOD ANALYTE COLLECTION DEVICE AND METHODS OF USE THEREOF

(71) Applicant: THE REGENTS OF THE UNIVERSITY OF CALIFORNIA, Oakland, CA (US)

(72) Inventors: Dorian Liepmann, Lafayette, CA (US); Jacobo Paredes, Berkeley, CA (US); Gerard Marriott, Palo Alto, CA (US)

(21) Appl. No.: 14/908,725

(22) PCT Filed: Aug. 21, 2014
(86) PCT No.: PCT/US14/52147

§ 371 Date: Jan. 29, 2016

Related U.S. Application Data
(60) Provisional application No. 61/869,008, filed on Aug. 22, 2013.

(51) Int. Cl. A61B 5/145; A61B 5/157; A61B 5/1468; A61B 5/15

(52) U.S. Cl. CPC A61B 5/14514; A61B 5/150984; A61B 5/157; A61B 5/14532; A61B 5/14546; A61B 5/1468; A61B 2010/008

(57) ABSTRACT

Provided is a blood analyte collection device that includes a microneedle array configured to provide fluid communication between a cellular interstitial fluid of a subject and a collection device fluid, a device chamber containing the collection device fluid, and a sequestration material in the device chamber configured to bind to a blood analyte from the cellular interstitial fluid. Also provided are methods and kits that use the subject blood analyte collection device. The subject devices, methods and kits find use in a variety of applications, such as detecting a blood analyte, such as glucose, in a subject.

### CROSS-REFERENCE TO RELATED APPLICATION

This application claims priority pursuant to 35 U.S.C. §119(e) to the filing date of U.S. Provisional Application No. 61/869,008, filed Aug. 22, 2013, the disclosure of which is incorporated herein by reference.

### INTRODUCTION

Blood analyte monitoring methods may be used for the diagnosis and treatment of various diseases. For example, blood glucose monitoring methods have been developed to aid in the care of both Type I diabetes and more recently Type II diabetes. Over the last decade, patients with Type II diabetes have increased in the United States and other countries. As a result, the market for self-monitoring of blood glucose (SMBG) now approaches $8.8 B per year. Currently, SMBG products typically require a blood sample for analysis, which may be obtained from a finger stick to produce a blood sample. However, blood samples obtained in this manner only provide a snapshot of a patient's immediate blood glucose levels.

Typical devices used to analyze these individual blood samples (such as the Roche Accu-Chek® glucose meter) can be used multiple times per day, but generally do not provide a daily average glucose level. More sophisticated blood glucose measurement methods may continuously monitor blood glucose levels in a patient, but require an implanted device and are typically used by patients with Type I diabetes. Examples of these devices are the Medtronic Revel, the MiniMed Paradigm and the Dexcom Seven Plus. However, continuous blood glucose monitoring devices still require regular finger-stick calibrations and an invasive implanted sensor.

In addition to monitoring blood analytes such as glucose, the concentration of HbA1C in the blood can be measured and is related to excess glucose levels over a two to three month timeframe. HbA1C tests are usually used to determine how well a patient with diabetes is controlling their blood glucose levels, and may also be used to diagnose diabetes. Similar to blood glucose tests described above, HbA1C tests typically require a blood sample for analysis. For example, some HbA1C test methods require a finger stick, while others may need a sample of blood from a vein.

### SUMMARY

Provided is a blood analyte collection device that includes a microneedle array configured to provide fluid communication between a cellular interstitial fluid of a subject and a collection device fluid, a device chamber containing the collection device fluid, and a sequestration material in the device chamber configured to sequester a blood analyte from the cellular interstitial fluid. Also provided are methods and kits that use the subject blood analyte collection device. The subject devices, methods and kits find use in a variety of applications, such as detecting a blood analyte, such as glucose, in a subject.

Aspects of the present disclosure include a blood analyte collection device that includes: (a) a microneedle array configured to provide fluid communication between a cellular interstitial fluid of a subject and a collection device fluid; (b) a device chamber containing the collection device fluid; and (c) a sequestration material in the device chamber configured to sequester a blood analyte from the cellular interstitial fluid. In some embodiments, the blood analyte is selected from sodium, potassium, urea, creatinine, glucose, HbA1C, chloride, calcium, ammonia, copper, phosphate, inorganic phosphorus, copper, zinc, magnesium, vitamin A, vitamin B₉, vitamin B₁₂, vitamin C, homocysteine, vitamin E, vitamin D, lead, ethanol, recreational drugs, lactate dehydrogenase, amylase, lipase, angiotensin-converting enzyme, acid phosphatase, eosinophil cationic protein, and a micronutrient, or mixtures thereof.

In some embodiments, the cellular interstitial fluid is present in the epidermis of the subject.

In some embodiments, the microneedle array includes microneedles having a length less than the thickness of the epidermis of the subject. In some embodiments, the microneedles have a length 50 μm to 200 μm. In some embodiments, the microneedles have a length 75 μm to 175 μm. In some embodiments, the microneedles have a length of 100 μm to 150 μm.

In some embodiments, the sequestration material includes a glucose binding protein. In some embodiments, the glucose binding protein is derived from Thermus thermophiles, Pseudomonas aeruginosa, Thermotoga maritime, Agrobacterium radiobacter, Pseudomonas aeruginosa, Lathyrus ochrus, pre-confluent chicken fibroblasts, confluent chicken fibroblasts, or mouse duodenal brush border membrane.

In some embodiments, the sequestration material comprises a molecularly imprinted polymer with glucose recognition sites.

Aspects of the present disclosure include a method for detecting a blood analyte in a subject. The method includes: (a) contacting the blood analyte collection device as described herein to a skin surface of a subject; (b) removing the blood analyte collection device from the skin surface of the subject; and (c) determining a concentration of the blood analyte.

In some embodiments, the determining includes: retrieving the sequestered blood analyte from the blood analyte collection device; and assessing the concentration of the blood analyte.

In some embodiments, multiple blood analytes are collected simultaneously.

In some embodiments, the blood analyte is selected from sodium, potassium, urea, creatinine, glucose, HbA1C, chloride, calcium, ammonia, copper, phosphate, inorganic phosphorus, copper, zinc, magnesium, vitamin A, vitamin B₉, vitamin B₁₂, vitamin C, homocysteine, vitamin E, vitamin D, lead, ethanol, recreational drugs, lactate dehydrogenase, amylase, lipase, angiotensin-converting enzyme, acid phosphatase, eosinophil cationic protein, and a micronutrient, or mixtures thereof. In some embodiments, the method further includes maintaining the blood analyte collection device on the skin surface of the subject for a period of time to collect the blood analyte. In some embodiments, the period of time is 2 hours to 96 hours. In some embodiments, the period of time is 5 hours to 60 hours. In some embodiments, the period of time is 12 hours to 48 hours. In some embodiments, the period of time is 24 hours.

Aspects of the present disclosure include a kit that includes a blood analyte collection device as described herein and a packaging containing the device.

### BRIEF DESCRIPTION OF THE DRAWINGS

FIG. 1 shows cross-sectional view of a blood analyte collection device according to embodiments of the present disclosure.

FIG. 2 shows cross-sectional detail of a single needle in the microarray of the blood analyte collection device positioned in the epidermis of a subject, according to embodiments of the present disclosure.

FIG. 3 shows a cross-sectional top view of the blood analyte collection device of FIG. 1, according to embodiments of the present disclosure.

FIG. 4 shows a cross-sectional top view of a blood analyte collection device of FIG. 1 several hours after device has been collecting analytes from a subject, according to embodiments of the present disclosure.

FIG. 5a to FIG. 5d shows a flow diagram, showing a time lapse of the blood analyte collection device of FIG. 1 (left) with the corresponding view of the device from FIG. 4 (right), according to embodiments of the present disclosure.

FIG. 6 shows a graph of the blood glucose levels of an early stage Type II diabetic patient as measured using a blood analyte collection device according to embodiments of the present disclosure.

FIG. 7a to FIG. 7e shows a flow diagram of the use and clinical applications of a blood analyte collection device, according to embodiments of the present disclosure.

FIG. 8A and FIG. 8B show a side view and perspective view, respectively, of an experimental setup to test a blood analyte collection device, according to embodiments of the present disclosure. FIG. 8C shows a bottom view of the same experimental setup.

FIG. 9 shows an enlarged top view of the experimental setup from FIG. 8A to FIG. 8C after a 24 hour diffusion period, according to embodiments of the present disclosure.

FIG. 10 shows an image of different sized membrane rings used in the experimental setup shown in FIG. 8A to 8C.

FIG. 11 shows a graph of normalized absorption of radial dye distribution vs. distance from center (mm) for a diffusion experiment, according to embodiments of the present disclosure.

FIG. 12A and FIG. 12B show images of a diffusion experiment, according to embodiments of the present disclosure.

### DETAILED DESCRIPTION

Provided is a blood analyte collection device that includes a microneedle array configured to provide fluid communication between a cellular interstitial fluid of a subject and a collection device fluid, a device chamber containing the collection device fluid, and a sequestration material in the device chamber configured to sequester a blood analyte from the cellular interstitial fluid. Also provided are methods and kits that use the subject blood analyte collection device. The subject devices, methods and kits find use in a variety of applications, such as detecting a blood analyte, such as glucose, in a subject.

Before the present invention is described in greater detail, it is to be understood that aspects of the present disclosure are not limited to the particular embodiments described, and as such may, of course, vary. It is also to be understood that the terminology used herein is for the purpose of describing particular embodiments only, and is not intended to be limiting, since the scope of embodiments of the present disclosure will be defined only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within embodiments of the present disclosure. The upper and lower limits of these smaller ranges may independently be included in the smaller ranges and are also encompassed within embodiments of the present disclosure, subject to any specifically excluded limit in the stated range. Where the stated range includes one or both of the limits, ranges excluding either or both of those included limits are also included in embodiments of the present disclosure.

Unless defined otherwise, all technical and scientific terms used herein have the same meaning as commonly understood by one of ordinary skill in the art to which this invention belongs. Although any methods and materials similar or equivalent to those described herein can also be used in the practice or testing of embodiments of the present disclosure, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that embodiments of the present disclosure are not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of embodiments of the present disclosure, embodiments of the devices for detecting a blood analyte in a subject are described first in greater detail. Following this description, methods of detecting a blood analyte and kits using the subject devices are provided. Finally, a review of the various applications in which the devices, methods, and kits may find use is provided.

### Devices

Provided is a blood analyte collection device that finds use in the collection of one or more blood analytes from a subject, such as blood analytes present in cellular interstitial fluid of a subject. Various aspects of the blood analyte collection device are described in the following sections.

### Microneedle Array

Embodiments of the blood analyte collection device include a microneedle array. The microneedle array is configured to provide fluid communication between cellular interstitial fluid of a subject, such as cellular interstitial fluid in the epidermis of a subject, and a fluid in the collection device (e.g., a collection device fluid). By “fluid communication” is meant that a fluid and/or analytes in the fluid may flow from one region to another region. For instance, fluid communication between cellular interstitial fluid of a subject and a collection device fluid provides for fluid flow and/or analyte diffusion between the cellular interstitial fluid of the subject and the collection device fluid. In these embodiments, fluid and/or analytes in the fluid may flow from the cellular interstitial fluid of the subject into the blood analyte collection device. In some instances, fluid and/or analytes in the fluid may flow from the blood analyte collection device into the cellular interstitial fluid of the subject.

Fluid communication between the cellular interstitial fluid and the collection device fluid may be established through a central lumen of a microneedle in the microneedle array. One or more of the microneedles in the microneedle array may include a central lumen that provides for fluid communication between the cellular interstitial fluid and the collection device fluid. The central lumen may have a proximal opening at one end of the lumen and a distal opening at the opposite end of the lumen. Proximal is meant nearer to the device, and distal is meant further away from the device. For instance, the proximal opening may provide an opening between the end of the microneedle attached to the device, and the distal opening may provide an opening at the opposing end of the microneedle (e.g., the end of the microneedle that contacts the skin of the subject). In certain embodiments, fluid communication between the cellular interstitial fluid and the collection device fluid is established by inserting the distal portion of the microneedle array through the stratum corneum of the skin of the subject such that the distal ends of the microneedles extend through the stratum corneum into the underlying epidermis. Cellular interstitial fluid in the epidermis of the subject may thus be in fluid communication with a collection device fluid via the central lumen of the microneedle as described above.

In certain embodiments, the microneedles of the microneedle array have a sufficient length to penetrate through the stratum corneum of the skin of the subject. For example, embodiments of the microneedle array include microneedles that have an average length of 10 μm or more, such as 20 μm or more, or 30 μm or more, or 40 μm or more, or 50 μm or more, or 60 μm or more, or 70 μm or more, or 80 μm or more, or 90 μm or more, or 100 μm or more, or 110 μm or more, or 120 μm or more, or 130 μm or more, or 140 μm or more, or 150 μm or more, or 160 μm or more, or 170 μm or more, or 180 μm or more, or 190 μm or more, or 200 μm or more. In some instances, the microneedle array includes microneedles that have an average length of 50 μm or more. By average is meant the arithmetic mean.

In certain embodiments, the microneedles of the microneedle array have a length sufficient to extend into the epidermis of the skin of the subject. In some instances, the microneedles of the microneedle array have a length sufficient to extend into the epidermis without extending through the epidermis into the dermis of the subject (e.g., the length of the microneedles is less than the thickness of the epidermis. In some instances, the microneedles of the microneedle array have a length such that when the microneedle array is applied to a skin surface of the subject, the distal ends of the microneedles are positioned in the epidermis of the subject (e.g., the distal ends of the microneedles penetrate through the stratum corneum into the epidermis, but do not penetrate all the way through the epidermis into the dermis). In some instances, the microneedles of the microneedle array have a length such that the distal ends of the microneedles are positioned in the epidermis and do not significantly contact blood vessels (e.g., capillaries) and/or nerves in the dermis. These embodiments may facilitate a minimization in bleeding and/or pain in the subject when the device is applied to the skin of the subject. In certain embodiments, the subject does not feel, or only slightly feels, the microneedles of the blood analyte collection device even when they are fully positioned in the epidermis. Because of this comfort level, the blood analyte collection device can be worn comfortably for extended periods of time (e.g., 24 hours or more).

For example, embodiments of the microneedle array include microneedles that have an average length of 250 μm or less, such as 240 μm or less, or 230 μm or less, or 220 μm or less, or 210 μm or less, or 200 μm or less, or 190 μm or less, or 180 μm or less, or 170 μm or less, or 160 μm or less, or 150 μm or less, or 140 μm or less, or 130 μm or less, or 120 μm or less, or 110 μm or less, or 100 μm or less, or 90 μm or less, or 80 μm or less, or 70 μm or less, or 60 μm or less, or 50 μm or less, or 40 μm or less, or 30 μm or less, or 20 μm or less, or 10 μm or less. In certain instances, the microneedle array includes microneedles that have an average length of 200 μm or less.

As such, in certain embodiments, the microneedle array includes microneedles that have an average length ranging from 10 μm to 250 μm, such as from 10 μm to 240 μm, or 20 μm to 230 μm, or 30 μm to 220 μm, or 40 μm to 210 μm, or 50 μm to 200 μm, or 60 μm to 190 μm, or 70 μm to 180 μm, or 75 μm to 175 μm, or 80 μm to 170 μm, or 90 μm to 160 μm, or 100 μm to 150 μm, or 110 μm to 140 μm, or 120 μm to 130 μm. For example, certain embodiments of the microneedle array include microneedles that have an average length ranging from 50 μm to 200 μm. Certain embodiments of the microneedle array include microneedles that have an average length ranging from 75 μm to 175 μm. Certain embodiments of the microneedle array include microneedles that have an average length ranging from 100 μm to 150 μm.

In certain embodiments, the microneedles are elongated microneedles, where the length of the microneedles is greater than the width of the microneedles. For instance, the ratio of the length of the microneedles to the width of the microneedles may be 2:1, or 3:1, or 4:1, or 5:1, or 6:1, or 7:1, or 8:1, or 9:1, or 10:1. In certain embodiments, the width (e.g., outside diameter) of the microneedles ranges from 1 μm to 100 μm, such as from 1 μm to 90 μm, or 1 μm to 80 μm, or 1 μm to 70 μm, or 1 μm to 60 μm, or 1 μm to 50 μm, or 1 μm to 40 μm, or 1 μm to 30 μm, or 1 μm to 20 μm, or 1 μm to 10 μm. In certain embodiments, the central lumen of the microneedles has a width (e.g., diameter) sufficient to allow for fluid communication of fluid and/or analytes in the fluid between the cellular interstitial fluid of the subject and the collection device fluid. For instance, the central lumen of the microneedles may have a width (e.g., diameter) of 0.1 μm to 50 μm, such as 0.5 μm to 40 μm, or 0.5 μm to 30 μm, or 0.5 μm to 20 μm, or 0.5 μm to 10 μm, or 0.5 μm to 7 μm, or 0.5 μm to 5 μm, or 0.5 μm to 3 μm, or 0.5 μm to 2 μm.

In certain embodiments, the microneedles have a shape, such as, but not limited to, a cylinder, a cone, a pyramid, and the like. In some cases, the cross-sectional profile of the microneedles is circular, elliptical, square, rectangular, and the like. In some instances, the tip of the microneedles is substantially flat, such that the tip of the microneedle is substantially parallel to the skin surface of the subject when the device is applied to the skin. In other embodiments, the tip of the microneedles may be angled with respect to the skin surface of the subject. In these embodiments, an angled tip may provide a smaller surface area that initially contacts the skin surface of the subject when the device is applied to the skin, which may facilitate penetration of the microneedles through the stratum corneum.

In certain embodiments, the microneedles have a central axis, such as a longitudinal axis through the central lumen of the elongated microneedles. In certain cases, the microneedles are substantially perpendicular to the substrate the microneedles are formed from and/or attached to, such that the longitudinal axis of the microneedles is substantially perpendicular to the substrate surface. In other embodiments, the microneedles are positioned at an angle with respect to the substrate surface, such as at an angle of less than 90 degrees between the longitudinal axis of the microneedles and the substrate surface. In some instances, microneedles are positioned at an angle with respect to the substrate surface may facilitate retention of the device in the skin of the subject.

The microneedles of the blood analyte collection device are provided in an array of multiple microneedles. For instance, a microneedle array may include 10 or more microneedles, 20 or more, 30 or more, 40 or more 50 or more, 60 or more, 70 or more, 80 or more, 90 or more, 100 or more, 150 or more, 200 or more, 250 or more, 300 or more, 400 or more, 500 or more, 600 or more, 700 or more, 800 or more, 900 or more, 1000 or more, 1500 or more, 2000 or more, 2500 or more, 3000 or more, 3500 or more, 4000 or more, 4500 or more, or 5000 or more microneedles in the microneedle array. The microneedles in the microneedle array may each be substantially the same as each other in terms of size and shape, as described above. In other embodiments, the size and shape of the microneedles in the microneedle array may vary, for example with different regions of the array containing microneedles of different sizes and/or shapes.

In certain embodiments, the microneedle array has an area ranging from 1 cm² to 100 cm², such as 1 cm² to 90 cm², or 1 cm² to 80 cm², or 1 cm² to 70 cm², or 1 cm² to 60 cm², or 1 cm² to 50 cm², or 1 cm² to 40 cm², or 1 cm² to 30 cm², or 1 cm² to 20 cm², or 1 cm² to 10 cm².

In certain instances, employing an array of microneedles, rather than a single microneedle, may facilitate a maximization in fluid contact between the cellular interstitial fluid and the blood analyte collection device fluid. The use of an array may also minimize the possibility of clogging the device.

The subject blood analyte collection device may include microneedle arrays as described by Stoeber et al. (U.S. Pat. No. 6,406,638), the disclosure of which is incorporated herein by reference. Other microneedle designs may also be appropriate for the blood analyte collection device. For example, the microneedles as described by Bisano, et. al. (U.S. Pat. No. 5,928,207), molded microneedles, as described by Talbot et. al. (U.S. Pat. No. 6,375,148), and hollow out-of-plain silicon needles, as described by Mukerjee et. al. (U.S. Pat. No. 7,753,888) may be used, the disclosures of each of which are incorporated herein by reference. Other microneedle types and uses include those described by Zimmermann et. al. (U.S. Pat. No. 7,415,299) and Jana (U.S. Pat. No. 8,280,476), the disclosures of each of which are incorporated herein by reference. The microneedles may be composed of metal and/or other materials, such as plastics or other polymers.

Microneedle array configurations, for example as described by Jina et al. (U.S. application Ser. No. 11/871,806), the disclosure of which is incorporated herein by reference, may also be used. Such considerations as length, diameter, material, tensile strength, flexibility, spacing, sharpness, and size of aperture will be apparent to the ordinarily skilled artisan, and may vary with the requirements of the material used, the production techniques employed, and the subject population using the device. For example, for children or elderly subjects who may have thinner skin, a shorter needle length may be used. For instance, the epidermis typically is from about 150 μm to 200 μm in depth below the skin surface, in some cases from about 100 μm to 170 μm in depth. The dermis containing capillaries and nerves may be from about 0.60 mm to 3 mm in depth, in some cases from about 1.50 mm to 3 mm. The selection of needle length may take into account the range of depths of these dermal structures. In some cases, such as children or infants, a shorter microneedle length may be utilized. Additionally, longer needles may be retrofitted with spacer elements to adapt them to an appropriate length.

### Device Chamber

Embodiments of the blood analyte collection device include a device chamber. The device chamber is configured to contain a collection device fluid. As described above, the blood analyte collection device is configured to provide fluid communication between the cellular interstitial fluid in the epidermis of the subject and the collection device fluid. As such, in these embodiments, the blood analyte collection device provides fluid communication between the cellular interstitial fluid of the subject and the interior volume of the device chamber, which contains the collection device fluid as described above. For example, the device chamber may include a surface that contacts the skin of the subject during use of the device. In some instances, the surface of the device chamber that contacts the skin of the subject includes a microneedle array. As described above, the microneedle array provides for fluid communication between the cellular interstitial fluid and the collection device fluid contained in the device chamber.

In certain embodiments, the device chamber has a volume ranging from 100 μL to 10 mL, such as 250 μL to 9 mL, or 500 μL to 8 mL, or 750 μL to 7 mL, or 1 mL to 5 mL, or 1 mL to 3 mL. In some instances, the device chamber has a length and width (e.g., diameter for circular shaped device chambers) ranging from 1 cm to 10 cm, such as 1 cm to 9 cm, or 1 cm to 8 cm, or 1 cm to 7 cm, or 1 cm to 6 cm, or 1 cm to 5 cm, or 1 cm to 4 cm, or 1 cm to 3 cm, or 1 cm to 2 cm. In certain cases, the device chamber has a thickness ranging from 100 μm to 2 cm, such as from 250 μm to 1.5 cm, or 500 μm to 1 cm, or 1 mm to 1 cm, or 2 mm to 1 cm, or 5 mm to 1 cm.

In certain embodiments, the device chamber contains a collection device fluid. In some instances, the collection device fluid is a fluid compatible with the analyte or analytes being collected by the blood analyte collection device. The collection device fluid may also be compatible with the materials the device is composed of, such as the device chamber material and the microneedle array material. In some embodiments, the collection device fluid is compatible with cellular interstitial fluid of the subject. For example, the collection device fluid may be isotonic with respect to the cells of the subject contacted by the collection device fluid, such as the cells of the epidermis of the subject. In some instances, the collection device fluid includes water (e.g., deionized water), saline, a buffer, combinations thereof, and the like.

Embodiments of the blood analyte collection device also include a sequestration material in the device chamber. The sequestration material is configured to bind to a blood analyte. Binding of the blood analyte to the sequestration material may include covalent bonds and non-covalent interactions, such as, but not limited to, ionic bonds, hydrophobic interactions, hydrogen bonds, van der Waals forces (e.g., London dispersion forces), dipole-dipole interactions, and the like. In some cases, binding of the blood analyte to the sequestration material sequesters the blood analyte from freely diffusing through the collection device fluid. Blood analytes bound to the sequestration material may be collected and analyzed to determine the presence and/or quantity of the blood analyte.

In certain instances, the blood analyte is sodium, potassium, urea, creatinine, glucose, HbA1C, chloride, calcium, ammonia, copper, phosphate, inorganic phosphorus, copper, zinc, magnesium, vitamin A, vitamin B₉, vitamin B₁₂, vitamin C, homocysteine, vitamin E, vitamin D, lead, ethanol, recreational drugs, lactate dehydrogenase, amylase, lipase, angiotensin-converting enzyme, acid phosphatase, eosinophil cationic protein, a micronutrient, or mixtures thereof. For example, the blood analyte may be glucose or HbA1C. As such, the sequestration material may be a material that specifically binds to one or more of the desired blood analytes to be detected and/or quantified.

In some instances, the blood analyte to be detected and/or quantified is glucose. In these embodiments, the sequestration material specifically binds to glucose. For example, the sequestration material may specifically bind to glucose present in the cellular interstitial fluid that is in fluid communication with the collection device fluid. In certain embodiments, the sequestration material includes a glucose, binding protein. Glucose binding proteins of interest include, but are not limited to, a glucose binding protein derived from Thermus thermophiles, Pseudomonas aeruginosa, Thermotoga maritime, Agrobacterium radiobacter, Pseudomonas aeruginosa, Lathyrus ochrus, pre-confluent chicken fibroblasts, confluent chicken fibroblasts, or mouse duodenal brush border membrane. Other sequestration materials that specifically bind glucose may also be used in the device, such as a glucose binding polymer. Glucose binding polymers include, but are not limited to, a glucose binding polymer with glucose recognition sites, e.g., a molecularly imprinted polymer with glucose recognition sites.

The blood analyte collection device can utilize any one or a combination of glucose binders and/or porous materials for glucose sequestration. By example, hydrogels can be employed to sequester glucose. In some cases, a standard hydrogel is cross-linked to produce a porous material. Glucose binding moieties, such as glucose binding proteins, may then incorporated into this porous structure. During the glucose collection process, these sequestration components bind glucose as described above.

In certain embodiments, a material with a high absorption coefficient may be used, such that the analyte of interest is bound substantially irreversibly. Polydimethylsiloxane (PDMS) is an example of such a material which can bind hydrophobic molecules. These high absorption coefficient materials may absorb analytes into their structure, and these absorbed analytes do not come back out, providing a partitioning effect. Plastics or other materials similar to PDMS but without its hydrophobicity character, are also suitable for the blood analyte collection device.

Glucose binding proteins (GBP) may be used in certain embodiments of the blood analyte collection device. GBPs do not alter the chemistry of glucose. Rather, the process is an equilibrium driven association between substrate (e.g., glucose) and protein. GBP and other binding proteins may retain their activity for longer periods in solution. GBP is in the periplasmic ligand binding protein family (PLBP), and is typically a monomeric periplasmic protein synthesized in the cytoplasm of various microorganisms and cell lines.

As an example, the GBP from Escherichia coli binds glucose with high affinity. Some examples of microbial sources of GBP are Thermus thermophiles, Pseudomonas aeruginosa, Thermotoga maritime, Agrobacterium radiobacter, Pseudomonas aeruginosa, and Lathyrus ochrus, among others. Some examples of animal sources of GBP are pre-confluent or confluent chicken fibroblasts, and mouse duodenal brush border membrane, among others. Various methods are available for purifying GBPs, such as described in PCT/GB2004/004907 and Edvotek 277 Affinity Chromatography of Glucose Binding Protein, and the like.

Molecularly imprinted polymers (MIPs) with glucose recognition sites may be used as a sequestration material in the blood analyte collection device. Molecularly imprinted polymer hydrogels displaying isomerically resolved glucose binding are described further by Wizeman et al. at the Department of Materials and Nuclear Engineering, University of Maryland, College Park, Md., USA.

### Methods

Aspects of the present disclosure include methods for detecting a blood analyte in a subject. The methods may be directed to determining whether a blood analyte is present in the cellular interstitial fluid of a subject, e.g., determining the presence or absence of one or more blood analytes in the subject. In certain embodiments of the methods, the presence of one or more blood analytes in the subject may be determined qualitatively or quantitatively. Qualitative determination includes determinations in which a simple yes/no result with respect to the presence of a blood analyte in the subject are determined. Quantitative determination includes both semi-quantitative determinations in which a rough scale result, e.g., low, medium, high, is determined regarding the amount of the blood analyte in the subject, and fine scale results in which an exact measurement of the concentration of the blood analyte is determined.

In certain embodiments, the blood analyte collection devices are used to detect the presence of one or more blood analytes in a subject. Blood analytes in a subject may be determined by sampling the cellular interstitial fluid of the subject, since the presence of a blood analyte in the cellular interstitial fluid may be directly proportional to the presence and/or concentration of the analyte in the blood flow of the subject. In some instances, the cellular interstitial fluid analyzed by the device is a complex sample. By “complex sample” is meant a sample that may or may not have the analytes of interest, but also includes many different proteins and other molecules that are not of interest. In some instances, the complex sample assayed in the subject methods is one that includes 10 or more, such as 20 or more, including 100 or more, e.g., 10³ or more, 10⁴ or more (such as 15,000; 20,000 or 25,000 or more) distinct (i.e., different) molecular entities. In certain instances, the blood analyte collection devices are capable of specifically binding one or more specific blood analytes from the complex sample of interstitial fluid.

Embodiments of the method of detecting a blood analyte in a subject include contacting a blood analyte collection device as described herein to a skin surface of a subject. The blood analyte collection device may be contact to the skin surface such that the microneedle array contacts the skin surface of the subject. In certain embodiments, the distal ends of the microneedles penetrate into the skin of the subject, for example through the stratum corneum and into the epidermis. Insertion of the distal ends of the microneedles into the epidermis establishes fluid communication between the cellular interstitial fluid in the epidermis and the collection device fluid.

In certain embodiments, the blood analyte collection device is maintained on the skin surface of the subject for a period of time. In some instances, fluid communication is maintained between the cellular interstitial fluid and the collection device fluid to allow collection of the blood analyte by the device. In certain cases, due to the fluid communication established between the cellular interstitial fluid and the collection device fluid contained in the device chamber, one or more blood analytes diffuses from the cellular interstitial fluid into the device chamber. In some instances, this fluid communication facilitates the establishment of a dynamic equilibrium between blood analytes in the subject's cellular interstitial fluid and the collection device fluid. Because the analyte concentration (e.g., glucose concentration) in the cellular interstitial fluid is similar or equal to that of the analyte concentration in the blood (e.g., blood glucose concentration), the blood analyte collection device may be used to determine the analyte concentration in the cellular interstitial fluid as an accurate surrogate for the analyte concentration in the blood of the subject.

For example, an embodiment of the blood analyte collection device may be used to collect blood glucose for the determination of blood glucose concentrations over a period of time. The blood analyte collection device may physically collect glucose from the blood without blood being directly sampled. This capability provided for by the diffusion of glucose from the cellular interstitial fluid into the collection device. The cellular interstitial fluid serves as the conduit of blood glucose to the cells of the body, which are the final destination for glucose in the body's physiological system. Thus, the cellular interstitial fluid comes from, and is a surrogate for the glucose levels in the neighboring blood capillaries, which feed glucose and other nutrients to the cells through the cellular interstitial fluid. The capillaries role in the body tissue is to transport nutrients out and into the cells, such as analytes and other materials, by active diffusion. The capillary beds are plentiful and are bathed in interstitial fluid. Not all the cells are in contact with the capillaries, so the cellular interstitial fluid acts as an inter-cellular medium that has the same or similar levels of analytes as the capillaries.

When the blood analyte collection device is first inserted into the skin surface of the subject, the analyte in the cellular interstitial fluid may diffuse into the fluid in the blood analyte collection device without the need for any external actuation. For example, no drawing of blood is required. As the analyte diffuses into the device chamber, the analyte to be detected may be sequestered (e.g., bound) by the sequestration material in the device. Sequestration of the analyte by the sequestration material removes the analyte from the diffusion equilibrium, allowing additional analyte to diffuse into the device. As such, a total analyte concentration over a period of time may be determined.

In certain embodiments, the period of time that the device is maintained on the skin surface of the subject (e.g., the blood analyte collection period) ranges from 2 hours to 96 hours, or 5 hours to 60 hours, or 12 hours to 48 hours, such as 24 hours. In some cases, the period of time that the device is maintained on the skin surface of the subject is 24 hours. While in some embodiments the collection period may be 24 hours, other collection periods may be employed depending on specific clinical needs. For instance, a shorter collection period may be appropriate for some blood analytes. In other cases, a specific period may be used, which takes into account diurnal rhythm, blood levels of analytes during exercise or sleep, etc. In certain embodiments, the device may be used to obtain a measurement of the blood analyte concentration at a single point in time, rather than over an extended period of time.

After the device has been maintained on the skin surface of the subject for the desired period of time, the method may further include removing the blood analyte collection device from the skin surface of the subject. Embodiments of the method further include determining the concentration of the desired blood analyte. For instance, determining the concentration of the blood analyte may include determining the concentration of the blood analyte that has been bound by the sequestration material in the device. The concentration of the blood analyte in the device may then be correlated to the concentration of analyte in the cellular interstitial fluid, and thus correlated to the concentration of the analyte in the blood of the subject. In certain embodiments, determining the concentration of the blood analyte includes retrieving the sequestered blood analyte from the blood analyte collection device and assessing the concentration of the blood analyte. In some instances, assessing the concentration of the blood analyte may be performed using standard analysis methods for determining the concentration of an analyte in a sample.

Once the desired collection period has elapsed, the blood analytes to be detected are stably engaged with the sequestration material. In some cases, the collection device can be sent by mail to a lab for analysis. Determination of the analyte concentration may be performed by a health care professional using standard laboratory blood analyte analysis systems. In some instances, the analyte concentration may be determined to provide a total blood analyte level (e.g., 24 hr total blood level). For example, blood glucose concentration may be determined to provide a blood glucose level (e.g., 24 hr total blood glucose level).

Embodiments of the methods provided herein may be used for the detection of glucose. However, glucose is just one example of a blood analyte that can be determined by the blood analyte collection device. For example, an adjunct to a glucose measurement may be to determine HbA1C levels concomitantly. Other analytes detected by the methods described herein can include sodium, potassium, urea, creatinine, glucose, chloride, calcium, ammonia, copper, phosphate, inorganic phosphorus, copper, zinc, magnesium, vitamin A, vitamin B₉, vitamin B₁₂, vitamin C, homocysteine, vitamin E, vitamin D, lead, ethanol, recreational drugs, lactate dehydrogenase, amylase, lipase, angiotensin-converting enzyme, acid phosphatase, eosinophil cationic protein, a micronutrient, and the like, and combinations thereof.

The subject methods may also be used for the simultaneous testing of multiple blood analytes. By example, two or more different sequestration materials may be used with each sequestration material specific to each analyte. As such, in some embodiments, multiple blood analytes may be collected simultaneously. Additionally, these sequestering materials can be placed in different segments of the device chamber. The individual segments of the device chamber may be separated and tested separately. Alternatively, the various analytes can be removed from the collection device en mass, and then tested using analysis methods for multiple analytes in the same sample.

### Diffusion Gradients and Pressures

The analyte (e.g., glucose) concentration may be subject to diffusion pressure. In some cases, the analyte (e.g., glucose) levels within the fluid of the blood analyte collection device may be substantially the same as those of the cellular interstitial fluid. As the analyte levels of interstitial fluid provide a surrogate to the analyte level in the blood, the total analyte concentration over a period of time (e.g., 24 hours) of the patient can thus be determined. In some embodiments of the blood analyte collection device, the system can be calibrated to take into account differences in analyte (e.g., glucose) diffusion rates and pressures that may be characteristic of the selected media in which the fluid of the blood analyte collection device will be contained.

Diffusion may be described by a power law, σᵣ²˜Dt^{α}, where D is the diffusion coefficient and t is the elapsed time. In a typical diffusion process, α=1. If α>1, the phenomenon is called super-diffusion. Super-diffusion can be the result of active cellular transport processes. If α<1, the particle undergoes sub-diffusion.

By example, anomalous diffusion is a diffusion process with a non-linear relationship to time, in contrast to a typical diffusion process, in which the mean squared displacement (MSD), σᵣ², of a particle is a linear function of time. Physically, the MSD can be considered the amount of space the particle has traversed in the system.

Anomalous diffusion may be used to describe physical scenarios, such as within crowded systems, for example protein diffusion within cells, or diffusion through porous media.

For diffusion in porous media the basic equations are:

$$J = - D ∇ {n}^{m}$$

$$\frac{\partial n}{\partial t} = D Δ {n}^{m}$$

where D is the diffusion coefficient, n is the concentration, m>0 (usually m>1, the case m=1 corresponds to Fick's law). These equations are appropriate for some of the porous gels which can be usefully employed in the blood analyte collection device.

The sequestration of analytes by the sequestration material generally occurs at a steady rate. Sequestration of the analyte may provide a profile of the patient's blood analyte (e.g., glucose) over time. Because the analyte (e.g., glucose) is sequestered from the dynamics of the diffusion equilibrium, the diffusion pressure between the interstitial fluid, the concentration in the blood analyte collection device fluid remains substantially constant over time. As a result, the analyte (e.g., glucose) from the interstitial fluid continues to diffuse into the fluid in the blood analyte collection device.

In certain embodiments, the method includes calibrating the device. For example, the device may be calibrated to account for patient dehydration or renal insufficiency, which may affect the relative levels of blood glucose levels in relation to those in the cellular interstitial fluid.

Embodiments of the devices and method are described further as they relate to the figures.

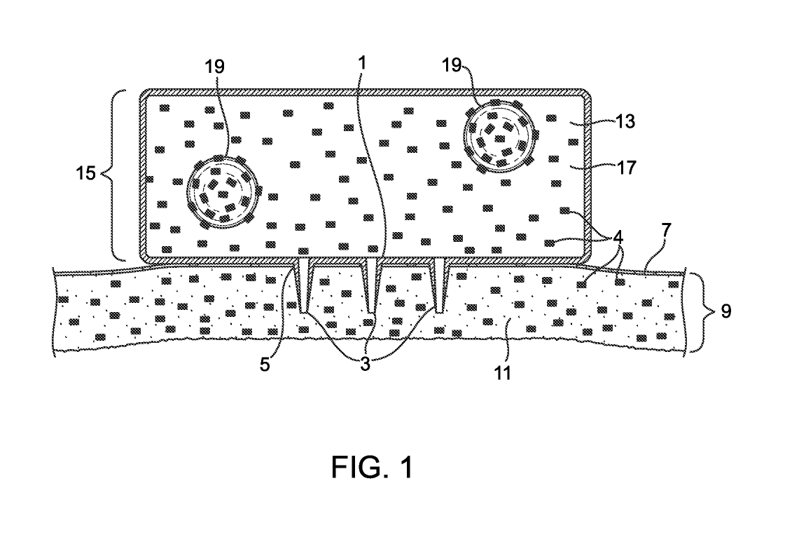

FIG. 1 shows an embodiment of the blood analyte collection device in cross section, as positioned on the skin in the process of collecting an analyte (e.g., glucose) for the assessment of a blood glucose level of the patient (e.g., a 24 hr total blood glucose level of the patient). In FIG. 1, microneedle array 1 is positioned on the skin of the test subject through simple finger pressure on the blood analyte collection device with sufficient force that the microneedles 3 form artificial pores 5 in skin surface stratum corneum 7 of the epidermis 9. In this manner, microneedle array 1 provides fluid communication between the patent's cellular interstitial fluid 11 within epidermis 9 with the device fluid 13 in device chamber 15. Once this fluid communication is established, diffusion of the analyte, in this case glucose 4, commences. In this way, a diffusion equilibrium is established between the glucose 4 in cellular interstitial fluid 11 and the glucose 4 in device fluid 13.

Within device chamber 15 is contained porous material 17 which is suffused with device fluid 13. Within porous material 17 is provided an analyte sequestration material, such as a glucose binding protein 19. Because this view is of the blood analyte collection device after a period of time for diffusion of glucose 4 has elapsed, the glucose 4 is contained within both the device chamber 15 and in the cellular interstitial fluid 11.

As the glucose binding proteins 19 bind glucose 4, the bound glucose is sequestered from the glucose diffusion system between the device fluid 13 and the cellular interstitial fluid 11. The glucose 4 which is bound to binding proteins 19 can be seen accumulating on the surface of binding proteins 19 in FIG. 1.

This accumulation and resultant sequestration of the glucose 4 continues as long as there are unbound glucose binding surfaces remaining on the surface of the glucose binding proteins 19 which are accessible to the glucose 4 remaining in solution. In this manner, glucose 4 will diffuse into the device fluid 13 from cellular interstitial fluid 11, but some of the glucose 4 will no longer be available to return to cellular interstitial fluid 11 by diffusion due to being bound to the glucose binding proteins 19.

As a part of the diffusion equilibrium of the glucose 4 remaining in solution, equilibrium is rapidly reestablished between the free glucose 4 in the device fluid 13 and cellular interstitial fluid 11. Because the body temperature at which the blood analyte collection device functions is within a specific range due to the body's homeostasis, the diffusion rate of the glucose 4 is substantially steady over a given period of time, and does not significantly vary in a manner that might otherwise confound the results.

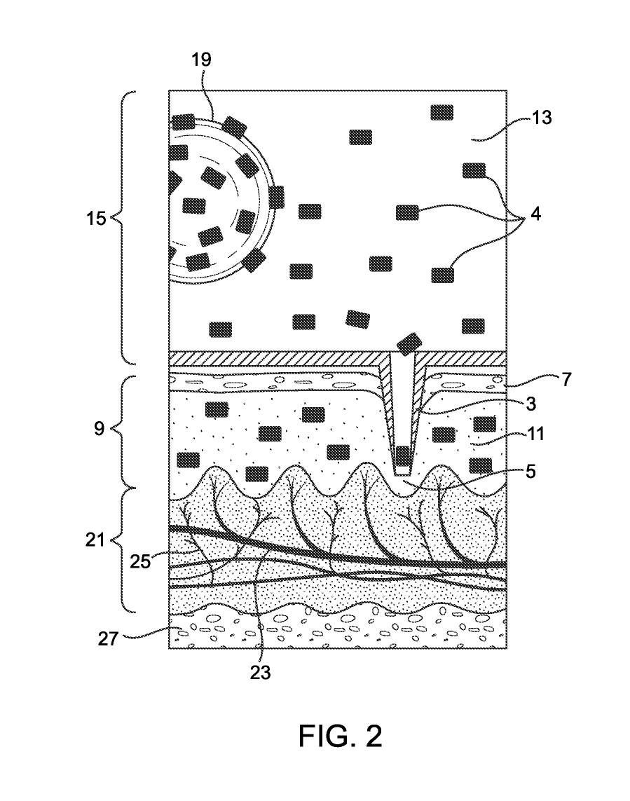

Referring now to FIG. 2, FIG. 2 provides a detailed view of one microneedle 3 and its channel 5 through epidermis 9 as shown in FIG. 1. Interstitial fluid 11 is shown within epidermis 9, which is in fluid communication with the device fluid 13 through channel 5 of microneedle 3 through the stratum corneum 7. The glucose 4 which is bound to binding proteins 19 can be seen accumulating on the surface of binding proteins 19.

FIG. 2 also shows the skin structures below epidermis 9, including dermis 21 that includes skin capillaries 23 and skin nerves 25. In some embodiments, microneedle 3 does not penetrate to skin nerves 25. As a result, the use of the blood analyte collection device does not cause significant pain or discomfort to the patent. Additionally, in some embodiments, microneedle 3 does not penetrate to skin capillaries 23. As such, in these embodiments, the blood analyte collection device does not cause significant bleeding, and minimizes the risk of potential blood contamination. Beneath dermis 21 lies subcutaneous fat 27.

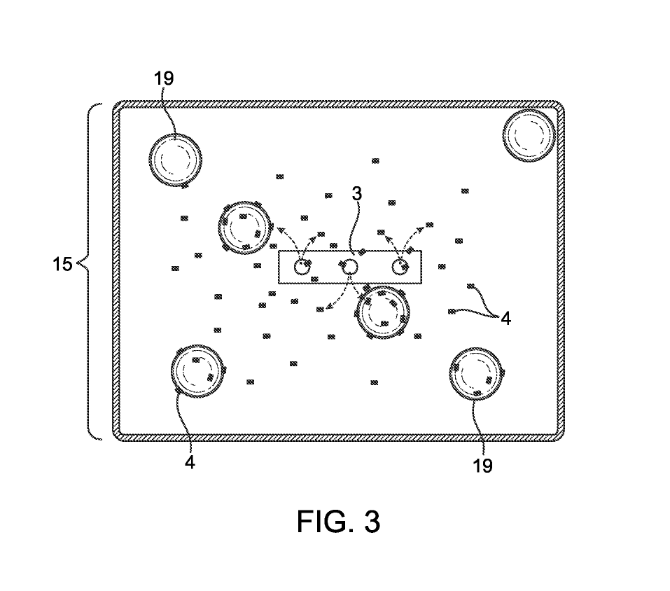

FIG. 3 is a top view of the blood analyte collection device of FIG. 1 and FIG. 2. In FIG. 3, glucose 4 enters into the device through microneedles 3 and is bound to the glucose binding proteins 19, such as the glucose binding proteins 19 adjacent to microneedles 3, which may be a result of interaction among the glucose 4 which is moving due to Brownian motion at standard body temperature. As shown in FIG. 3, the glucose 4 binds to the first binding proteins 19 to which they come into contact when diffusing through the device fluid. As the binding proteins 19 closest to the microneedles 3 become saturated with glucose 4, additional glucose 4 entering into the device through microneedles 3 will no longer have the opportunity to bind to the saturated binding sites. In addition, with steric hindrance and a smaller available binding surface, it is also less likely that additional glucose 4 will bind to other binding sites on the binding proteins 19 most adjacent to microneedles 3. Therefore, additional glucose 4 diffusing though the device are more likely to bind to binding proteins 19 farther from the microneedles 3.

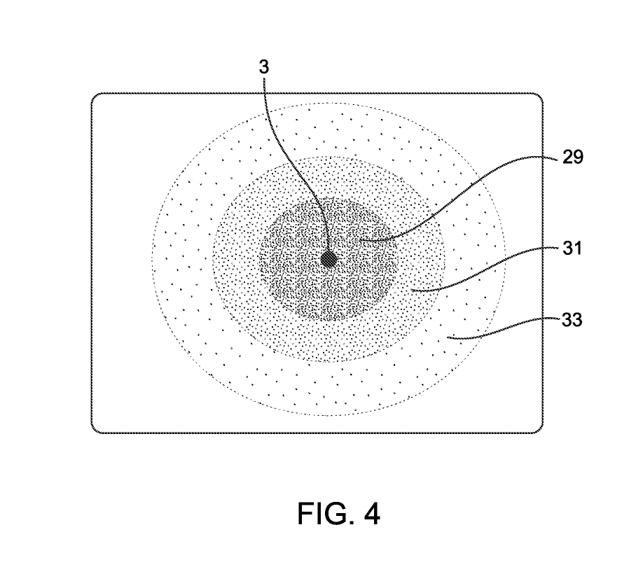

FIG. 4 is a top view of the blood analyte collection device of FIG. 1 and FIG. 2 shown with resulting glucose binding over time. FIG. 4 shows a microneedle array 3 with an area 29 around the microneedles. As the subject wears the blood analyte collection device, initially the glucose will bind in an initial binding area 29 adjacent the microneedles 3. After a period of time, as the binding proteins 19 (not shown) in initial binding area 29 become saturated with glucose (shown in FIG. 4 as the shading in area 29), additional glucose entering the device will spread to a second binding area 31. As the subject continues to wears the blood analyte collection device, and depending on how much blood glucose the subject has, the binding areas will continue to expand over time, such as from third binding area 33 outwards. Because the glucose is sequestered by the glucose binding proteins, the diffusion pressure differential between the interstitial fluid and the fluid in the device remains substantially the same. As a result, the glucose will continue to enter into the blood analyte collection device, unless all glucose binding protein binding sites becomes saturated and the diffusion pressure differential equalizes.

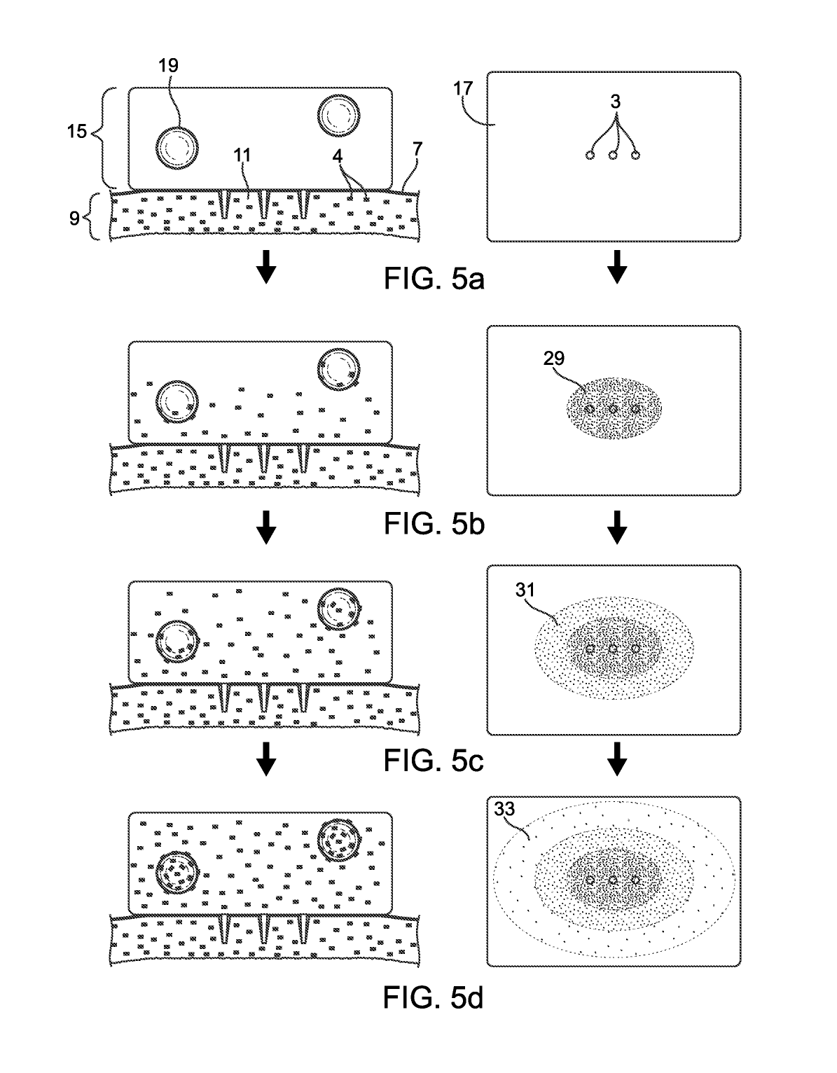

FIG. 5 is a flow diagram of the blood analyte collection device as shown during use. The left column provides cross-sectional views similar to that shown in FIG. 1. The right column provides the corresponding top views of the blood analyte collection device, as shown dynamically in FIG. 4. Referring first to FIG. 5a, FIG. 5a represents the blood analyte collection device when it is initially placed on the subject's skin, and channels are produced in epidermis 9 by microneedles 3 penetrating the stratum corneum 7. In FIG. 5a, glucose 4 has not yet entered into the device chamber 15 from cellular interstitial fluid 11. The corresponding top view in FIG. 5a shows 3 microneedles 3, and the porous material 17 in the device chamber which is suffused with device fluid. Within porous material 17 is provided a glucose sequestration material, such as a glucose binding protein 19.

Referring now to view FIG. 5b, the glucose 4 is beginning to enter into device chamber 15 through microneedles 3, with some glucose binding to binding proteins 19. In the corresponding top view in FIG. 5b, glucose is entering the device chamber through the microneedles with some glucose binding to the binding proteins closest to microneedles 3, such as the binding proteins in a first binding area 29.

Referring now to view FIG. 5c, after additional glucose binding to binding proteins 19, the bound glucose 4 has substantially saturated the binding proteins 19 in first binding area 29. Without significant binding opportunities in binding area 29, the free glucose 4 begins to diffuse to second binding area 31 and bind to binding proteins 19 in that area.

Referring now to view FIG. 5d, after additional glucose binding to binding proteins 19, the glucose 4 in the cellular interstitial fluid 11 is substantially in equilibrium with the unbound glucose particles 4 in device chamber 15. Without substantial binding opportunities in binding area 29 and binding area 31, the free glucose 4 begins to diffuse to binding area 33 and bind to binding proteins 19 in that area. As demonstrated, this producing a glucose gradient or tree-ring type effect, providing additional information regarding the rate of glucose migration into the blood analyte collection device over time.

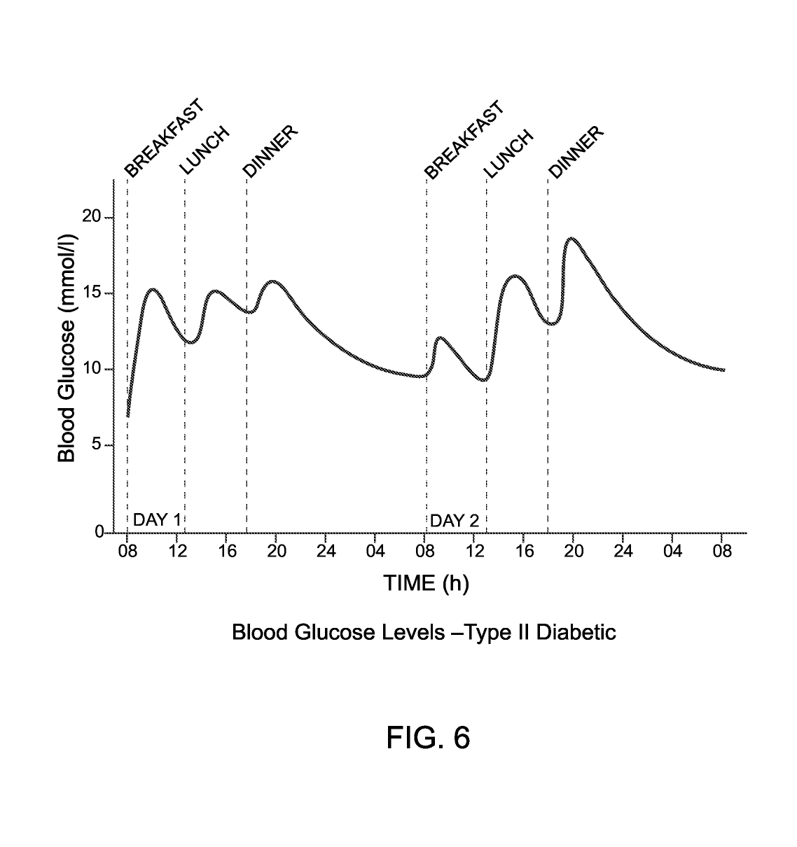

FIG. 6 is a graph showing blood glucose levels in a type II diabetic patent. In some instances, there may be high variability of glucose levels over the course of a day. Additionally, it can be seen that this variability may be different from day to day, even at the same time of day and with the same general timing of activities. As such, single point glucose assessment methods may not provide an accurate determination of a patient's total blood levels over an extended period of time, such as over 24 hours. In certain embodiments, the blood analyte collection device provides an accurate clinical assessment of a patient's total blood glucose level over an extended period of time (e.g., 24 hours) regardless of blood glucose variability over time.

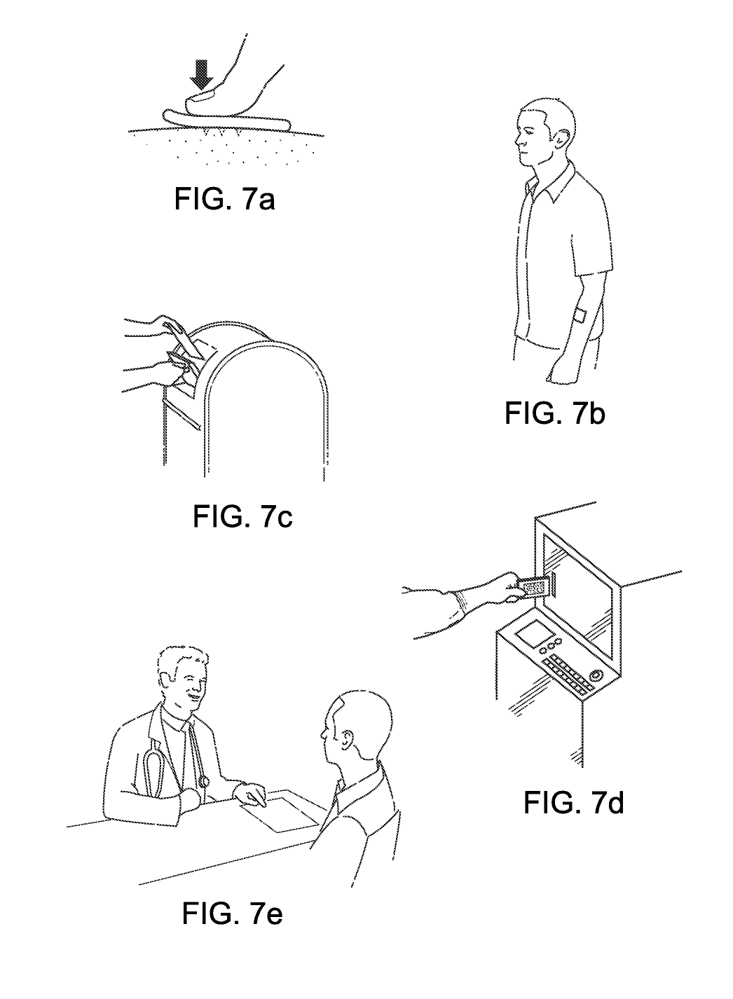

FIG. 7a to FIG. 7e show a graphic illustration of the use of a blood analyte collection device by a patient. FIG. 7a shows the placement of a blood analyte collection device on the patient's skin. While in this view it is simply pressed onto the skin surface by finger pressure, other methods can be employed, such applying pressure with a heavy object. Once positioned on the skin, the blood analyte collection device can be maintained in place for the duration of the testing period. In some cases, the blood analyte collection device may be maintained in place by applying an adhesive bandage over the blood analyte collection device. In certain cases, an elastic wrap can be used to secure the blood analyte collection device, such as for individuals who are sensitive to adhesives.

In some cases, the blood analyte collection device is placed on the subject's skin by a health care professional, such as at a doctor's office. In some cases, the skin may be cleaned before applying the device so that there is not undue surface material which might interfere with the collection processes. For example, the surface of the skin may be swabbed with an alcohol or other antibacterial solution. This may serve both to clean the skin area and dissolve away possible oils of other materials on the surface of the skin. The blood analyte collection device may then be applied to the skin and optionally covered with a protective bandage, as described above. A protective bandage may be used such that the device stays in position during testing, and is protected against possible dirt or fluid contamination during the testing period.

FIG. 7b shows the subject going about their daily activities while wearing the blood analyte collection device. Also, as described above, the blood analyte collection device can be worn intermittently. In this way, key times of the patient's day, or key activities can be selected for monitoring. For instance, sleep periods, periods of time following sleep periods, post-meal times, pre-meal times, periods of exercise, periods of time following exercise, and the like. Differences in blood glucose levels between weekend schedules and workday schedules can also be assessed.

FIG. 7c shows that at the end of an assessment period (e.g., 24 hours), the test subject removes the blood analyte collection device, and in this case places it in an envelope to send to the test laboratory for analysis. Other approaches to removing and delivering the device are possible. For example, when patient compliance is a concern, the device may be removed at a doctor's office or clinic. In these embodiments, there may be increased assurance that the device was in skin contact throughout the testing period, and that the test area was not subject to undue disturbance. In addition, a health care professional may check the status of the overlying bandage material, if used. In other embodiments, the blood analyte collection device can be returned directly to the health care professional for analysis by the health care professional.

FIG. 7d shows the blood analyte collection device being tested. In certain embodiments, the contents of the device chamber are dissolved and analyzed. Standard blood analyte testing equipment may be used.

In certain embodiments, after the appropriate collection time period, for instance 24 hours, the contents of the device chamber (e.g., the hydrogel and fluid in the device chamber) are either removed and dissolved, or directly dissolved in the device. In certain embodiments, the concentration of the glucose in the device chamber is proportional to the average concentration of glucose in the subject's cellular interstitial fluid during the analysis period.

FIG. 7e shows a health care professional, having received the total blood glucose test results, conferring with the patient on a treatment plan.

Although the above examples are described in relation to the collection and detection of blood glucose, the examples and descriptions provided herein also apply to the collection and detection of other blood analytes as described herein.

### Utility

The subject devices and methods find use in a variety of different applications where determination of the presence or absence, and/or quantification of one or more blood analytes in a subject is desired. In certain embodiments, the methods are directed to the detection of a blood analyte, such as blood glucose, in a subject. The methods may include the detection of a set of blood analytes, e.g., two or more distinct blood analytes, in a subject. For example, the methods may be used in the rapid, clinical detection of two or more blood analytes in a subject, e.g., as may be employed in the diagnosis of a disease condition in the subject, in the ongoing management or treatment of a disease condition in the subject, etc.

In certain embodiments, the subject devices and methods find use in detecting blood analytes that may be used as biomarkers. In some cases, the subject devices and methods may be used to detect the presence or absence of particular biomarkers, as well as an increase or decrease in the concentration of particular biomarkers in cellular interstitial fluid in a subject.

The presence or absence of a biomarker or significant changes in the concentration of a biomarker can be used to diagnose disease risk, presence of disease in an individual, or to tailor treatments for the disease in an individual. For example, the presence of a particular biomarker or panel of biomarkers may influence the choices of drug treatment or administration regimes given to an individual. In evaluating potential drug therapies, a biomarker may be used as a surrogate for a natural endpoint such as survival or irreversible morbidity. If a treatment alters the biomarker, which has a direct connection to improved health, the biomarker can serve as a surrogate endpoint for evaluating the clinical benefit of a particular treatment or administration regime. Thus, personalized diagnosis and treatment based on the particular biomarkers or panel of biomarkers detected in an individual are facilitated by the subject devices, systems and methods. Furthermore, the early detection of biomarkers associated with diseases is facilitated by the high sensitivity of the subject devices.

The subject devices and methods find use in diagnostic assays, such as, but not limited to, the following: detecting and/or quantifying biomarkers, as described above; screening assays, where samples are tested at regular intervals for asymptomatic subjects; prognostic assays, where the presence and or quantity of a biomarker is used to predict a likely disease course; stratification assays, where a subject's response to different drug treatments can be predicted; efficacy assays, where the efficacy of a drug treatment is monitored; and the like.

The subject devices, systems and methods also find use in validation assays. For example, validation assays may be used to validate or confirm that a potential disease biomarker is a reliable indicator of the presence or absence of a disease across a variety of individuals.

The subject devices and methods find use in the determination of a total 24 hr blood glucose level for patients. In some instances, a total 24 hr blood glucose level test may improve treatment of the diabetic population by enabling the accurate diagnosis and monitoring of diabetes, such as Type II diabetes. This diagnosis in early stage Type II diabetes may facilitate lifestyle changes that can be started before the patient's cells become more intransigently insulin insensitive or resistant.

Currently the standard test for determining blood glucose is a fasting blood glucose test. This test requires the patent to abstain from eating for an extended period of time (e.g., overnight). This requirement of the test may be difficult for pregnant women, teenagers, children and the elderly. At the point of testing, a bolus of glucose is ingested that may be unappetizing, again difficult for pregnant women and the elderly. Blood is then drawn over a two hour period, as that is generally the tolerance of the test subject to remain in a clinical environment under these conditions. For current blood glucose assessment methods, a single or otherwise limited blood test typical of practical clinical environments, the information is analogous to throwing a dart at a dart board. The points of blood collection may be susceptible to overestimates or underestimates of total blood glucose levels over an extended period of time (e.g., 24 hrs). Confounding the current single point testing available is that patent compliance with pre-testing fasting requirements may be inconsistent, and patient reporting of same may often be unreliable. Also, even with consistent reporting, individuals may vary considerably in their body's response to a meal, effects of varying exercise, hormones, and other confounding factors.

The subject blood analyte collection device finds use as a complete and reliable clinical assessment of a total 24 hr blood glucose level for a patient, regardless of their blood glucose variability over time. In addition, as described above, several analytes may be tested in a single blood analyte collection device. Because the microneedle array penetrates into the epidermis without contacting the blood vessels and/or nerves in the underlying dermis, the subject devices find use in the collection and determination of a blood analyte without drawing blood from a subject. In some instances, this may facilitate a reduction in pain of a blood draw through a hypodermic needle. In some cases, this may facilitate the treatment of patients adverse to the use of needles for finger stick and/or venous blood glucose measurements, who may otherwise forgo glucose testing.

As described above, in some embodiments, the blood analyte collection device may be sent to a remote testing facility, which performs the analysis for the blood analytes collected by the device. In some instances, the opportunity for a patient to receive the blood analyte collection device by mail, and return the device by mail after blood analyte collection may facilitate the convenience of blood analyte testing, for example in a rural medical setting. The components of the blood analyte collection device may be mechanically and chemically stable, such that the device can be sealed in an envelope and mailed to a remote test site. This convenience of testing enabled by the blood analyte collection device may facilitate blood analyte testing for elderly patients with limited mobility.

The blood analyte collection device and methods find use for a continuous assessment of total blood analyte (e.g., glucose) levels over a set time period. During the time period that the device is applied to the skin of a subject, the blood analyte collection device may be similar in comfort and convenience to wearing a bandage. For those patients with sensitivity to adhesives, the device can be secured to the skin by a bandage or an elastic strap.

In certain embodiments, the blood analyte collection device and methods find use in the detection of blood analytes typically present in blood at very low concentrations. For example, due the integration over an extended period of time (e.g., 24 hrs), the device facilitates testing of low concentration blood analytes which may be difficult to detect in a blood sample. In some instances, the subject devices can detect low concentration blood analytes through accumulation in the collection device, thus providing a clinically practical analyte level determination, such as for the analysis of micronutrients and trace chemicals.

### Kits

Aspects of the present disclosure additionally include kits that have a device as described in detail herein. In some instances, the kit includes a packaging for containing the device. In certain embodiments, the packaging may be a sealed packaging, e.g., in a water vapor-resistant container, optionally under an air-tight and/or vacuum seal. In certain instances, the packaging is a sterile packaging, configured to maintain the device enclosed in the packaging in a sterile environment. By “sterile” is meant that there are substantially no microbes (such as fungi, bacteria, viruses, spore forms, etc.).

In certain embodiments, the kit includes one or more devices as described herein. For example, a kit may include 2 or more devices, such as 3 or more, or 4 or more, or 5 or more, or 6 or more, or 7 or more devices, including 10 or more, or 14 or more, or 21 or more or 28 or more, or 30 or more devices. In some cases, a kit includes 7 devices as described herein. In some cases, a kit includes 14 devices as described herein. In some cases, a kit includes 30 devices as described herein. In some embodiments of the kit that include 2 or more devices, each device may be provided in an individual packaging (e.g., an individual sterile packaging) as described above. In some embodiments of the kit that include 2 or more devices, the devices may be provided in a packaging that includes 2 or more compartments, where each compartment contains a single device and is configured to maintain the device enclosed in the compartment in a sterile environment. In these embodiments, each compartment may be opened individually as needed to obtain a device from within the opened compartment without disrupting the sterile environment of the remaining unopened compartments.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. In some embodiments, the instructions may be provided on a computer readable medium, e.g., CD, DVD, Blu-Ray, computer-readable memory (e.g., flash memory), etc., on which the information has been recorded or stored. In some embodiments, the instructions may be provided as a website address which may be used via the Internet to access the information at a removed site. Any convenient method for providing instructions to the user may be present in the kits.

As can be appreciated from the disclosure provided above, embodiments of the present devices and methods have a wide variety of applications. Accordingly, the examples presented herein are offered for illustration purposes and are not intended to be construed as a limitation on the invention in any way. Those of ordinary skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### EXAMPLES

### Example 1

Experiments were performed to test diffusion into a device as disclosed herein.

### Experimental Setup

A syringe was attached to the underside of a petri dish. The interior volume of the syringe was 2 mL. To attach the syringe to the petri dish, a Luer lock syringe was used and threaded into a hole in the bottom of the petri dish. The hole in the petri dish was tapped with a tapered thread. Teflon tape was used to seal the syringe to the petri dish to minimize leaks. 0.2 wt % (5 mM) fluorescein dye solution in deionized (DI) water was loaded into the syringe. A piece of Watman filter paper, presoaked in DI water, was placed on the top side of the petri dish over the hole such that the end of the Luer lock fitting was in contact with the diffusion membrane. A top plate was clamped onto the membrane to prevent interstitial space and side wall diffusion.

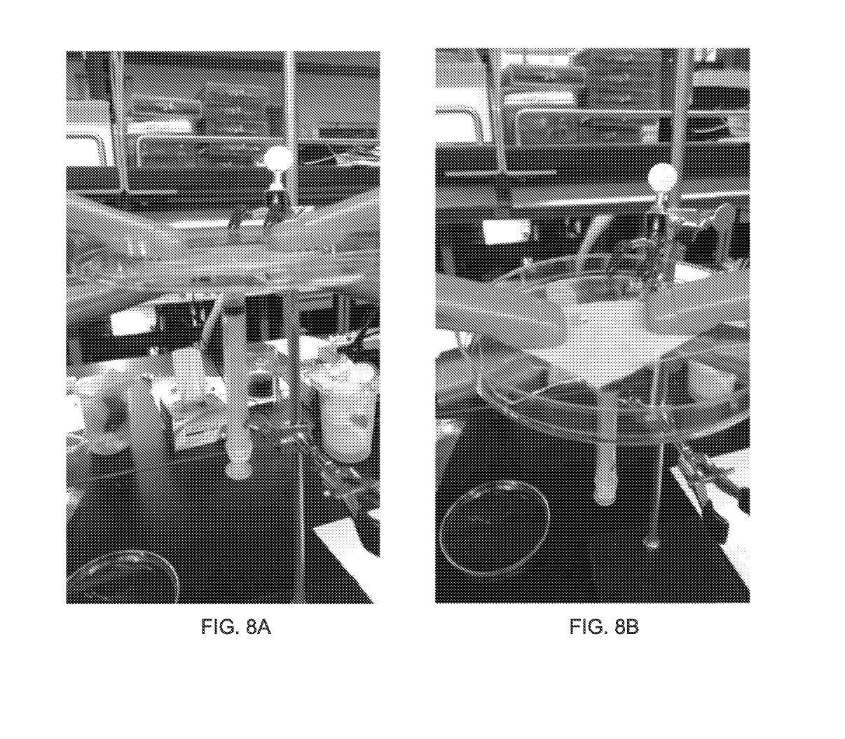

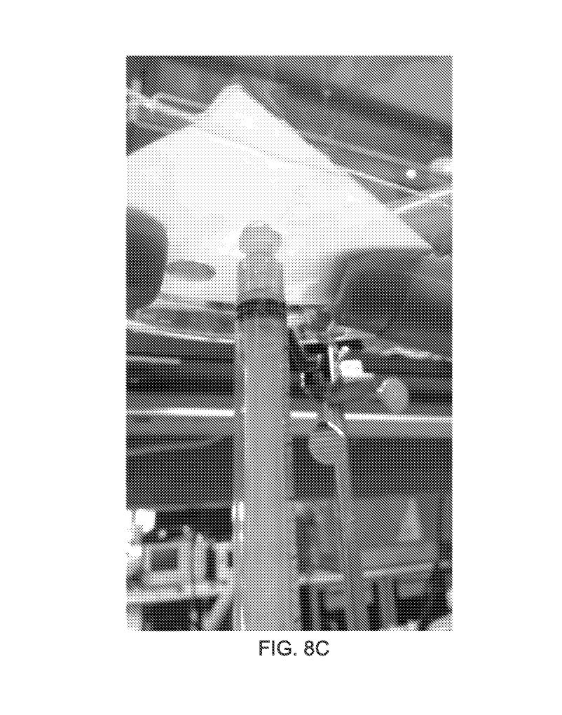

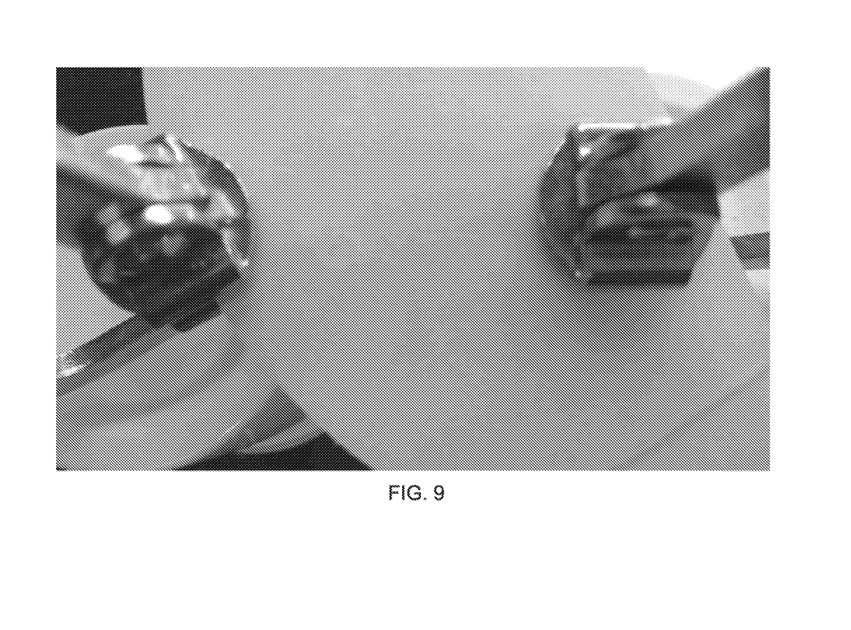

FIG. 8A and FIG. 8B show a side view and perspective view, respectively, of an experimental setup to test diffusion into a blood analyte collection device. FIG. 8C shows a bottom view of the same experimental setup.

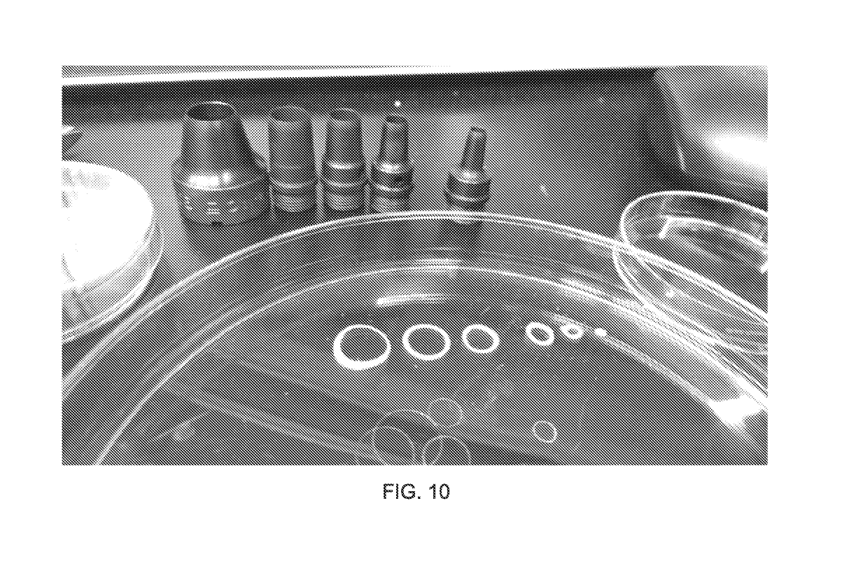

Membrane rings were cut using a punch. Different sized membrane rings were tested, ranging from 2 mm to 12 mm. See FIG. 10. The membrane ring was dried, first with the top plate in place, and then without the top plate, to prevent wicking of fluorescein dye across the membrane. Each membrane ring was soaked in 20 μL of DI water in a 96 well plate, with each ring in a different well. Bordering wells were filled with DI water to keep water from evaporating out of the test wells. The rings were soaked overnight to reach equilibrium, and kept under foil to minimize photobleaching. The limit of detection corresponded to the edge of the test area via UV-Vis detection.

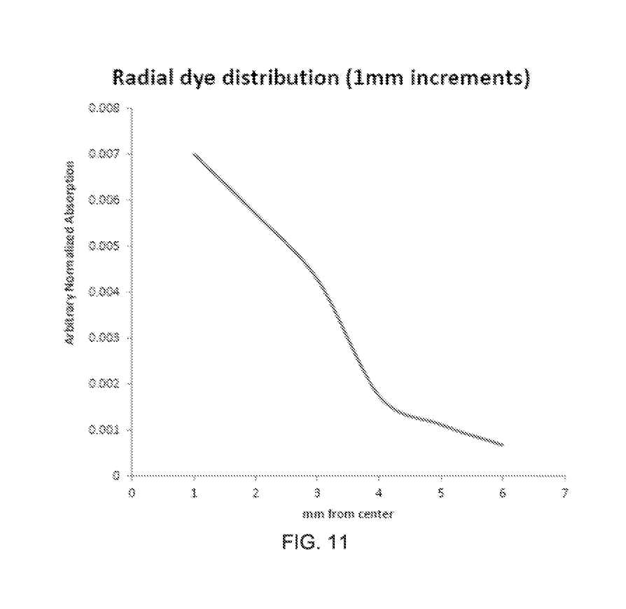

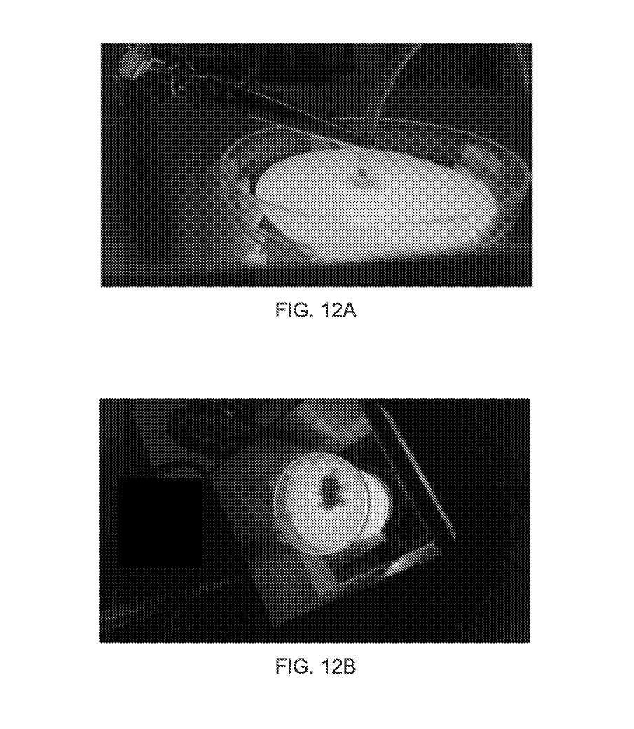

1.5 μL of solution in each well was assayed after aspiration. Equilibrium was assumed between the absorbed and free state of the dye. The membrane was kept wet over the 24 our test period. The top plate left a portion of the membrane exposed, such that the exposed portion of the membrane could wick in water that was on the plate. FIG. 9 shows an enlarged top view of the experimental setup from FIG. 8A to FIG. 8C after a 24 hour diffusion period, according to embodiments of the present disclosure. FIG. 11 shows a graph of normalized absorption of radial dye distribution vs. distance from center (mm) for a diffusion experiment, according to embodiments of the present disclosure. The slight dip in the absorption curve may be due to pressure placed on the membrane from the threaded fitting protruding out into the membrane. FIG. 12A and FIG. 12B show images of a diffusion experiment. Diffusion of the dye can be seen as the dark colored area.

The preceding merely illustrates the principles of embodiments of the present disclosure. It will be appreciated that those skilled in the art will be able to devise various arrangements which, although not explicitly described or shown herein, embody the principles of embodiments of the present disclosure and are included within its spirit and scope. Furthermore, all examples and conditional language recited herein are principally intended to aid the reader in understanding the principles of embodiments of the present disclosure and the concepts contributed by the inventors to furthering the art, and are to be construed as being without limitation to such specifically recited examples and conditions. Moreover, all statements herein reciting principles, aspects, and embodiments of the present disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of embodiments of the present disclosure, therefore, is not intended to be limited to the exemplary embodiments shown and described herein. Rather, the scope and spirit of present invention is embodied by the appended claims.

That which is claimed is:

1. A blood analyte collection device comprising:
(a) a microneedle array configured to provide fluid communication between a cellular interstitial fluid of a subject and a collection device fluid;
(b) a device chamber containing the collection device fluid; and
(c) a sequestration material in the device chamber configured to bind to a blood analyte from the cellular interstitial fluid.

2. The blood analyte collection device of claim 1, wherein the blood analyte is selected from the group consisting of sodium, potassium, urea, creatinine, glucose, HbA1C, chloride, calcium, ammonia, copper, phosphate, inorganic phosphorus, copper, zinc, magnesium, vitamin A, vitamin B₉, vitamin B₁₂, vitamin C, homocysteine, vitamin E, vitamin D, lead, ethanol, recreational drugs, lactate dehydrogenase, amylase, lipase, angiotensin-converting enzyme, acid phosphatase, eosinophil cationic protein, and a micronutrient, or mixtures thereof.

3. The blood analyte collection device of claim 1, wherein the cellular interstitial fluid is present in the epidermis of the subject.

4. The blood analyte collection device of claim 1, wherein the microneedle array comprises microneedles having a length less than the thickness of the epidermis of the subject.

5. The blood analyte collection device of claim 4, wherein the microneedles have a length 50 μm to 200 μm.

6. The blood analyte collection device of claim 4, wherein the microneedles have a length 75 μm to 175 μm.

7. The blood analyte collection device of claim 4, wherein the microneedles have a length of 100 μm to 150 μm.

8. The blood analyte collection device of claim 1, wherein the sequestration material comprises a glucose binding protein.

9. The blood analyte collection device of claim 8, wherein the glucose binding protein is derived from Thermus thermophiles, Pseudomonas aeruginosa, Thermotoga maritime, Agrobacterium radiobacter, Pseudomonas aeruginosa, Lathyrus ochrus, pre-confluent chicken fibroblasts, confluent chicken fibroblasts, or mouse duodenal brush border membrane.

10. The blood analyte collection device of claim 1, wherein the sequestration material comprises a glucose binding polymer.

11. A method for detecting a blood analyte in a subject, the method comprising:
(a) contacting the blood analyte collection device of claim 1 to a skin surface of a subject;
(b) removing the blood analyte collection device from the skin surface of the subject; and
(c) determining a concentration of the blood analyte.

12. The method of claim 11, wherein the determining comprises:
retrieving the bound blood analyte from the blood analyte collection device; and
assessing the concentration of the blood analyte.

13. The method of claim 11, wherein multiple blood analytes are collected simultaneously.

14. The method of claim 11, wherein the blood analyte is selected from the group consisting of sodium, potassium, urea, creatinine, glucose, HbA1C, chloride, calcium, ammonia, copper, phosphate, inorganic phosphorus, copper, zinc, magnesium, vitamin A, vitamin B₉, vitamin B₁₂, vitamin C, homocysteine, vitamin E, vitamin D, lead, ethanol, recreational drugs, lactate dehydrogenase, amylase, lipase, angiotensin-converting enzyme, acid phosphatase, eosinophil cationic protein, and a micronutrient, or mixtures thereof.

15. The method of claim 11, further comprising maintaining the blood analyte collection device on the skin surface of the subject for a period of time to collect the blood analyte.

16. The method of claim 15, wherein the period of time is 2 hours to 96 hours.

17. The method of claim 15, wherein the period of time is 5 hours to 60 hours.

18. The method of claim 15, wherein the period of time is 12 hours to 48 hours.

19. The method of claim 15, wherein the period of time is 24 hours.

20. A kit comprising:
analyte collection device of claim 1; and
a packaging containing the device.
